(19) United States
(12) Patent Application Publication
(10) Pub. No.: US 2007/0098805 A1
(43) Pub. Date: May 3, 2007

(54) Methods of making and using novel griseofulvin compositions

(75) Inventor: Gary G. Liversidge, West Chester, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/546,378

(22) Filed: Oct. 12, 2006

Related U.S. Application Data
(62) Division of application No. 10/683,154, filed on Oct. 14, 2003.
Application No. 10/683,154 is a continuation-in-part of application No. 10/175,851, filed on Jun. 21, 2002, now abandoned.
Application No. 10/175,851 is a continuation of application No. 08/815,346, filed on Mar. 11, 1997, now Pat. No. 6,432,381.

(51) Int. Cl. A61K 31/343; A61K 9/14

(52) U.S. Cl. 424/489; 514/462; 977/906

(57) ABSTRACT

The present invention is directed to nanoparticulate compositions comprising griseofulvin. The griseofulvin particles of the composition preferably have an effective average particle size of less than about 2 microns.

### CROSS-REFERENCE TO RELATED APPLICATIONS

This application is a divisional application of U.S. application Ser. No. 10/683,154, filed Oct. 14, 2003 (pending), which is a continuation-in-part of U.S. application Ser. No. 10/175,851, filed on Jun. 21, 2002 (abandoned), which is a continuation of U.S. application Ser. No. 08/815,346, filed on Mar. 11, 1997, now U.S. Pat. No. 6,432,381.

### FIELD OF THE INVENTION

The present invention relates to a novel compositions of griseofulvin, comprising griseofulvin particles having an effective average particle size of less than about 2000 nm and at least one surface stabilizer.

### BACKGROUND OF THE INVENTION

I. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having associated with the surface thereof a non-crosslinked surface stabilizer. The '684 patent does not describe nanoparticulate griseofulvin compositions.

Methods of making nanoparticulate active agent compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.” These patents do not describe methods of making nanoparticulate griseofulvin.

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide)Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat No. 6,375,986 for “Solid Dose Nanoparticulate Compositions Comprising a Synergistic Combination of a Polymeric Surface Stabilizer and Dioctyl Sodium Sulfosuccinate,” U.S. Pat. No. 6,428,814 for “Bioadhesive nanoparticulate compositions having cationic surface stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” and U.S. Pat. No. 6,592,903 for “Nanoparticulate Dispersions Comprising a Synergistic Combination of a Polymeric Surface Stabilizer and Dioctyl Sodium Sulfosuccinate,” all of which are specifically incorporated by reference. In addition, U.S. Patent Application No. 20020012675 A1, published on Jan. 31, 2002, for “Controlled Release Nanoparticulate Compositions,” and WO 02/098565 for “System and Method for Milling Materials,” describe nanoparticulate active agent compositions, and are specifically incorporated by reference. None of these references describe nanoparticulate griseofulvin compositions.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” These references do not describe nanoparticulate griseofulvin.

II. Background Regarding Griseofulvin

Griseofulvin is an antifungal antibiotic first isolated from a Penicillium species in 1939. The compound is insoluble in water, and slightly soluble in ethanol, methanol, acetone, benzene, CHCl₃, ethyl acetate, and acetic acid. Griseofulvin has the chemical formula C₁₇H₁₇ClO₆ and the following chemical structure: 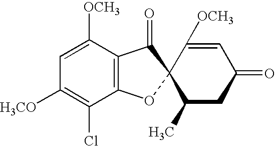
See The Merck Index, 10^{th} Edition, pp. 653-654 (1983).

Griseofulvin is given orally to treat dermatophyte and ringworm infections of the scalp, hair, nails (fingernails and toenails), and skin caused by specific fungi. Griseofulvin is used to treat skin infections such as jock itch, athlete's foot, and ringworm. In particular, griseofulvin is useful in treating Tinea capitis (ringworm of the scalp), Tinea corporis (ringworm of the body), Tinea pedis (athlete's foot), Tinea unguium (ringworm of the nails), Tinea cruris (ringworm of the thigh), and Tinea barbae (barber's itch). Griseofulvin is also used to the following fungal infections of the hair, skin, and nails: Trichophyton rubrum, Trichophyton tonsurans, Trichophyton mentagrophytes, Trichophyton interdigitalis, Trichophyton verrucosum, Trichophyton sulphureum, Trichophyton schoenleini, Trichophyton audouini, Trichophyton canis, Trichophyton gypseum, Trichophyton floccosum, Trichophyton megnini, Trichophyton gallinae, and Trichophyton crateriform. This medication is sometimes prescribed for other uses, and it is used as an antifungal agent in veterinary medicine. Griseofulvin may be taken alone or used along with medicines that are applied to the skin for fungus infections.

Griseofulvin stops fungal cells dividing (i.e., it is fungistatic) but does not kill them outright. This means treatment needs to be continued for several weeks or months. Griseofulvin is generally well tolerated in children.

Griseofulvin is available as a tablet, capsule, and liquid to take by mouth. It is usually taken once a day or can be taken two to four times a day. Griseofulvin is usually taken for 2 to 4 weeks for skin infections, 4 to 6 weeks for hair and scalp infections, 4 to 8 weeks for foot infections, 3 to 4 months for fingernail infections, and at least 6 months for toenail infections.

The dose of griseofulvin will be different for different patients. In general, for adults the dose is 500 mg to 1 g daily, and for children it is 10-25 mg per kg body weight per day.

Griseofulvin absorption from the gastrointestinal tract varies considerably among individuals, mainly because of insolubility of the drug in aqueous media of the upper GI tract. Physician's Desk Reference, 57^{th} Edition, p. 2445 (2003). The peak serum level found in fasting adults given 0.5 g occurs at about 4 hours and ranges between 0.5 and 2.0 mcg/mL.

Thus, griseofulvin is not very well absorbed from the gut. The drug should be taken after a meal or drink of milk as fat increases the absorption. The medication is carried into the skin by sweat and within a couple of weeks is concentrated in the outer skin layers.

Half the medication is cleared from the blood stream in 10 to 20 hours; the rest is eliminated in urine and faeces. This means the medication can be taken once daily. Griseofulvin should be continued until the fungal infection has completely gone because the medication is quickly cleared from skin and hair when it is stopped. Side effects of griseofulvin include headache; gastrointestinal upset, including nausea, vomiting, heartburn, cramps, flatulence, taste disturbance, diarrhea or loose stools, and furred tongue; thirst; fever; sore throat; skin rash (increased sun sensitivity); mouth soreness or irritation; urinary disturbance, including increased frequency and bed wetting; nervous system disturbance, including blurred vision, dizziness, depression, nightmares, faintness, and fatigue; menstrual disturbance; and liver disturbance.

Griseofulvin has exhibited interactions with dietary supplements. In particular, adding 50 IU of vitamin E per day was reported to increase blood levels of griseofulvin within four weeks in children, allowing the drug dose to be cut in half. Reducing the amount of griseofulvin should decrease the likelihood of side effects.

Griseofulvin is currently produced by different companies. The microcystalline form is marketed as Grifulvin V™ (Ortho Dermatological). (Fulvicin U/F™ (Schering) and Grisactin™ (Wyeth-Ayerst), the other two microcrystalline products, are no longer marketed.) The ultramicrocrystalline form of griseofulvin is marketed as Gris-PEG™ (Pedinol). (Fulvicin P/G™ (Schering) and Grisactin Ultra™ (Wyeth-Ayerst), the other two ultramicrocrystalline products, are no longer marketed.)

U.S. Pat. No. 6,604,698 for “Media Milling,” issued on Aug. 12, 2003, describes a process for preparing a dispersion of solid particles of a milled substrate, which can be griseofulvin, in a fluid carrier. The method comprises: (a) providing a plurality of large size milling media to the milling chamber of a media mill and forming a depth filter on an exit screen or separator in the milling chamber; (b) adding to the milling chamber a plurality of small size milling media, a conglomerate of a solid substance comprising a substrate to be milled, such as griseofulvin, and optionally one or more than one surface active substance, and a fluid carrier; (c) milling the conglomerate in the milling chamber to produce very small milled substrate product particles; and (d) continuously removing or separating the milled substrate particles suspended in the fluid carrier from the media through the depth filter. The media are retained in the milling chamber.

U.S. Pat. No. 6,569,463, for “Solid carriers for improved delivery of hydrophobic active ingredients in pharmaceutical compositions” issued on May 27, 2003, describes pharmaceutical compositions in the form of a solid carrier comprising a substrate and an encapsulation coat on the substrate. The encapsulation coat comprises an admixture of a therapeutically effective amount of a hydrophobic pharmaceutical active ingredient, such as griseofulvin, an effective solubilizing amount of at least one hydrophilic surfactant, and a lipophilic additive selected from the group consisting of lipophilic surfactants, triglycerides, and combinations thereof. The effective solubilizing amount of the at least one hydrophilic surfactant is an amount effective to partially or fully solubilize the pharmaceutical active ingredient in the encapsulation coat. This process is undesirable as it requires solubilizing the active agent, which can change the properties of the active agent. In addition, the solvents required to solubilize the active agent can have undesirable side effects.

U.S. Pat. No. 5,785,976 for “Solid lipid particles, particles of bioactive agents and methods for the manufacture and use thereof,” issued on Jul. 28, 1998, describes a process for making solid lipid particles (SLPs). The process comprises: (a) melting a solid agent, such as griseofulvin; (b) heating a dispersion medium to approximately the same temperature as the molten solid agent; (c) adding one or more highly mobile water-soluble or dispersible stabilizers to the dispersion medium in such a way that the amount of highly mobile stabilizers is, after emulsification, sufficient to stabilize newly created surfaces during recrystallization; (d) homogenizing the melted agent and dispersion medium by high-pressure homogenization, micro-fluidization and/or ultrasonication; and (d) cooling the homogenized dispersion until solid particles are formed by recrystallization of the dispersed agents. This process is undesirable as it requires melting the active agent, which can change the properties of the active agent.

U.S. Pat. Nos. 5,449,521 and 5,354,560, both for “Supported drugs with increased dissolution rate, and a process for their preparation,” issued on Sep. 12, 1995, and Oct. 11, 1994, respectively, describe supported drugs having an increased dissolution rate and prepared by a process comprising mixing the drug with the support material under dry conditions, co-grinding the mixture in a mill with its grinding chamber saturated with the vapour of one or more solvents able to solubilize the drug or to be adsorbed on the surface of the support material, vacuum-drying the product obtained, and sieving. The drugs obtained in this manner have a reduced heat of fusion, a reduced melting point, an increased dissolution rate and an increased solubilization kinetics. This process is undesirable as it requires solubilizing the active agent, which can change the properties of the active agent. In addition, the solvents required to solubilize the active agent can have undesirable side effects.

Finally, U.S. Pat. No. 5,705,194 for “Pharmaceutical compositions containing polyalkylene block copolymers which gel at physiological temperature,” issued on Jan. 6, 1998, describes a pharmaceutical composition which gels at physiological temperature. The composition comprises a triblock copolymer containing one or more polyoxyethylene blocks and one or more polyoxy (higher alkylene) blocks, wherein at least some of the blocks are linked together by a linking group characterized in that the linking group is an oxymethylene group, and a therapeutic agent. The therapeutic agent, which can be griseofulvin, is present as: (a) particles having an average size of less than about 400 nm and having the block copolymer adsorbed on the surface thereof, (b) a suspension in a solution of the block copolymer, or (c) as an aqueous solution in a solution of the block copolymer.

At concentrations as low as 2.5% w/v in phosphate balance salt solution (PBS) or in water, the described block copolymers have gel points close to physiological temperature (37.4° C.). The viscosity of these block copolymers at 3.5% and 5.5% in PBS changes abruptly from less than 20 cps at room temperature to more than 1500 cps at physiological temperature, while the pH and osmolality of the block copolymer solutions remain comparable to PBS. Thus, compositions containing these block copolymers can be administered (e.g., subcutaneously or orally) as low viscosity compositions at room temperature and, when they reach physiological temperature, will tend to gel.

Disadvantages of composition including such polyalkylene block copolymers include potential problems with IV administration, as well as potential difficulties in formulating the nanoparticulate composition into dosage forms for administration.

There is a need in the art for griseofulvin compositions which can improve clinical efficacy, reduce fed/fasted variability, and potentially reduce side effects. The present invention satisfies these needs.

### SUMMARY OF THE INVENTION

The present invention relates to nanoparticulate griseofulvin compositions. The compositions comprise griseofulvin and at least one surface stabilizer. The nanoparticulate griseofulvin particles have an effective average particle size of less than about 2 microns.

Another aspect of the invention is directed to pharmaceutical compositions comprising a nanoparticulate griseofulvin composition of the invention. The pharmaceutical compositions preferably comprise griseofulvin, at least one surface stabilizer, and at least one pharmaceutically acceptable carrier, as well as any desired excipients. Advantages and properties of the compositions of the invention are described herein.

The invention further discloses a method of making a nanoparticulate griseofulvin composition. Such a method comprises contacting griseofulvin and at least one surface stabilizer for a time and under conditions sufficient to provide a nanoparticulate griseofulvin composition. The one or more surface stabilizers can be contacted with griseofulvin either before, preferably during, or after size reduction of the griseofulvin.

The present invention is also directed to methods of treating fungal infections using the nanoparticulate griseofulvin compositions. Such infections include, for example, such as dermatophyte and ringworm infections of the scalp, hair, nails (fingernails and toenails), and skin caused by specific fungi.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to nanoparticulate griseofulvin compositions. The compositions comprise griseofulvin and at least one surface stabilizer that is preferably adsorbed on or associated with the surface of the drug. The nanoparticulate griseofulvin particles have an effective average particle size of less than about 2 microns.

As taught in the '684 patent, not every combination of surface stabilizer and active agent will result in a stable nanoparticulate composition. It was surprisingly discovered that stable nanoparticulate griseofulvin formulations can be made.

The current formulations of griseofulvin suffer from the following problems: (1) the poor solubility of the drug results in a relatively low bioavailability; (2) there is a significant variability in the fed/fasted absorption of the drug; (3) a wide variety of side effects are associated with the current dosage forms of the drug.

The present invention overcomes problems encountered with the prior art griseofulvin formulations. Specifically, the nanoparticulate griseofulvin formulations of the invention may offer the following advantages as compared to prior griseofulvin compositions: (1) faster onset of action; (2) a potential decrease in the frequency of dosing; (3) smaller doses of griseofulvin required to obtain the same pharmacological effect; (4) increased bioavailability; (5) an increased rate of dissolution; (6) improved performance characteristics for oral, intravenous, subcutaneous, or intramuscular injection, such as higher dose loading and smaller tablet or liquid dose volumes; (7) improved pharmacokinetic profiles, such as improved Tₘₐₓ, C^{max}, and AUC profiles; (8) substantially similar or bioequivalent pharmacokinetic profiles of the nanoparticulate griseofulvin compositions when administered in the fed versus the fasted state; (9) bioadhesive griseofulvin formulations, which can coat the gut or the desired site of application and be retained for a period of time, thereby increasing the efficacy of the drug as well as eliminating or decreasing the frequency of dosing; (10) high redispersibility of the nanoparticulate griseofulvin particles present in the compositions of the invention following administration; (11) the nanoparticulate griseofulvin compositions can be formulated in a dried form which readily redisperses; (12) low viscosity liquid nanoparticulate griseofulvin dosage forms can be made; (13) for liquid nanoparticulate griseofulvin compositions having a low viscosity—better subject compliance due to the perception of a lighter formulation which is easier to consume and digest; (14) for liquid nanoparticulate griseofulvin compositions having a low viscosity—ease of dispensing because one can use a cup or a syringe; (15) the nanoparticulate griseofulvin compositions can be used in conjunction with other active agents; (16) the nanoparticulate griseofulvin compositions can be sterile filtered; (17) the nanoparticulate griseofulvin compositions are suitable for parenteral administration; and (18) the nanoparticulate griseofulvin compositions do not require organic solvents or pH extremes.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules.

The dosage form of the invention can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

“Conventional” or “non-nanoparticulate active agent” shall mean an active agent which is solubilized or which has an effective average particle size of greater than about 2 microns. Nanoparticulate active agents as defined herein have an effective average particle size of less than about 2 microns.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Poorly water soluble drugs” as used herein means those having a solubility of less than about 30 mg/ml, preferably less than about 20 mg/ml, preferably less than about 10 mg/ml, or preferably less than about 1 mg/ml. Such drugs tend to be eliminated from the gastrointestinal tract before being absorbed into the circulation.

As used herein with reference to stable drug particles, “stable” includes, but is not limited to, one or more of the following parameters: (1) that the griseofulvin particles do not appreciably flocculate or agglomerate due to interparticle attractive forces, or otherwise significantly increase in particle size over time; (2) that the physical structure of the griseofulvin particles is not altered over time, such as by conversion from an amorphous phase to crystalline phase; (3) that the griseofulvin particles are chemically stable; and/or (4) where the griseofulvin has not been subject to a heating step at or above the melting point of the griseofulvin in the preparation of the nanoparticles of the invention.

“Therapeutically effective amount” as used herein with respect to a drug dosage, shall mean that dosage that provides the specific pharmacological response for which the drug is administered in a significant number of subjects in need of such treatment. It is emphasized that “therapeutically effective amount,” administered to a particular subject in a particular instance will not always be effective in treating the diseases described herein, even though such dosage is deemed a ‘therapeutically effective amount’ by those skilled in the art. It is to be further understood that drug dosages are, in particular instances, measured as oral dosages, or with reference to drug levels as measured in blood.

I. Preferred Characteristics of the Nanoparticulate Griseofulvin Compositions of the Invention

A. Increased Bioavailability, Frequency of Dosing, and Dosage Quantity

The nanoparticulate griseofulvin compositions of the invention may preferably exhibit increased bioavailability and require smaller doses as compared to prior non-nanoparticulate griseofulvin compositions administered at the same dose.

Any drug, including griseofulvin, can have adverse side effects. Thus, lower doses of griseofulvin that can achieve the same or better therapeutic effects as those observed with larger doses of non-nanoparticulate griseofulvin compositions are desired. Such lower doses may be realized with the nanoparticulate griseofulvin compositions of the invention because the nanoparticulate griseofulvin compositions may exhibit greater bioavailability as compared to non-nanoparticulate griseofulvin formulations, which means that smaller doses of griseofulvin are likely required to obtain the desired therapeutic effect.

The griseofulvin compositions of the invention may be administered less frequently and at lower doses in dosage forms such as liquid dispersions, powders, sprays, solid re-dispersable dosage forms, ointments, creams, etc. Exemplary types of formulations useful in the present invention include, but are not limited to, liquid dispersions, gels, aerosols (pulmonary and nasal), ointments, creams, solid dose forms, etc. of nanoparticulate griseofulvin. Lower dosages can be used because the small particle size of the griseofulvin particles ensure greater absorption, and in the case of bioadhesive nanoparticulate griseofulvin compositions, the griseofulvin is retained at the desired site of application for a longer period of time as compared to conventional griseofulvin dosage forms.

In one embodiment of the invention, the therapeutically effective amount of the nanoparticulate griseofulvin compositions is ⅙, ⅕, ¼, ⅓^{rd}, or ½ of the therapeutically effective amount of a non-nanoparticulate griseofulvin composition.

Such lower doses are preferred as they may decrease or eliminate adverse effects of the drug. In addition, such lower doses decrease the cost of the dosage form and may increase patient compliance.

B. Pharmacokinetic Profiles of the Nanoparticulate Griseofulvin Compositions of the Invention

The invention also preferably provides griseofulvin compositions having a desirable pharmacokinetic profile when administered to mammalian subjects. The desirable pharmacokinetic profile of the griseofulvin compositions preferably includes, but is not limited to: (1) a Tₘₐₓ for griseofulvin, when assayed in the plasma of a mammalian subject following administration, that is preferably less than the Tₘₐₓ for a non-nanoparticulate griseofulvin formulation administered at the same dosage; (2) a Cₘₐₓ for griseofulvin, when assayed in the plasma of a mammalian subject following administration, that is preferably greater than the Cₘₐₓ for a non-nanoparticulate griseofulvin formulation administered at the same dosage; and/or (3) an AUC for griseofulvin, when assayed in the plasma of a mammalian subject following administration, that is preferably greater than the AUC for a non-nanoparticulate griseofulvin formulation administered at the same dosage.

The desirable pharmacokinetic profile, as used herein, is the pharmacokinetic profile measured after the initial dose of griseofulvin. The compositions can be formulated in any way as described below and as known to those of skill in the art.

A preferred griseofulvin composition of the invention exhibits in comparative pharmacokinetic testing with a non-nanoparticulate griseofulvin formulation administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate griseofulvin formulation.

This shorter Tₘₐₓ translates into a faster onset of therapeutic activity. The use of conventional formulations of griseofulvin is not ideal due to delayed onset of action. Specifically, conventional griseofulvin formulations exhibit a peak plasma concentration at 4 hours following administration. In contrast, the nanoparticulate griseofulvin compositions of the invention exhibit faster therapeutic effects.

A preferred griseofulvin composition of the invention exhibits in comparative pharmacokinetic testing with a non-nanoparticulate griseofulvin formulation of administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cmax exhibited by the non-nanoparticulate griseofulvin formulation.

A preferred griseofulvin composition of the invention exhibits in comparative pharmacokinetic testing with a non-nanoparticulate griseofulvin formulation administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate griseofulvin formulation.

Any formulation giving the desired pharmacokinetic profile is suitable for administration according to the present methods. Exemplary types of formulations giving such profiles are liquid dispersions, gels, aerosols, ointments, creams, solid dose forms, etc. of nanoparticulate griseofulvin.

C. The Pharmacokinetic Profiles of the Nanoparticulate Griseofulvin Compositions of the Invention are Preferably not Substantially Affected by the Fed or Fasted State of the Subject Ingesting the Compositions

The invention encompasses nanoparticulate griseofulvin compositions wherein preferably the pharmacokinetic profile of the griseofulvin is not substantially affected by the fed or fasted state of a subject ingesting the composition. This means that there is no substantial difference in the quantity of griseofulvin absorbed or the rate of griseofulvin absorption when the nanoparticulate griseofulvin compositions are administered in the fed versus the fasted state. Thus, the nanoparticulate griseofulvin compositions of the invention can substantially eliminate the effect of food on the pharmacokinetics of griseofulvin.

In another embodiment of the invention, the pharmacokinetic profile of the griseofulvin compositions of the invention, when administered to a mammal in a fasted state, is bioequivalent to the pharmacokinetic profile of the same griseofulvin composition administered at the same dosage, when administered to a mammal in a fed state. “Bioequivalency” is preferably established by a 90% Confidence Interval (CI) of between 0.80 and 1.25 for both Cₘₐₓ and AUC under U.S. Food and Drug Administration (USFDA) regulatory guidelines, or a 90% CI for AUC of between 0.80 to 1.25 and a 90% CI for Cmax of between 0.70 to 1.43 under the European Medicines Evaluation Agency (EMEA) regulatory guidelines (Tₘₐₓ is not relevant for bioequivalency determinations under USFDA and EMEA regulatory guidelines).

Preferably the difference in AUC (e.g., absorption) of the nanoparticulate griseofulvin composition of the invention, when administered in the fed versus the fasted state, is less than about 100%, less than about 90%, less than about 80%, less than about 70%, less than about 60%, less than about 50%, less than about 40%, less than about 35%, less than about 30%, less than about 25%, less than about 20%, less than about 15%, less than about 10%, less than about 5%, or less than about 3%.

In addition, preferably the difference in Cₘₐₓ of the nanoparticulate griseofulvin composition of the invention, when administered in the fed versus the fasted state, is less than about 100%, less than about 90%, less than about 80%, less than about 70%, less than about 60%, less than about 50%, less than about 40%, less than about 35%, less than about 30%, less than about 25%, less than about 20%, less than about 15%, less than about 10%, less than about 5%, or less than about 3%.

Finally, preferably the difference in the Tₘₐₓ of the nanoparticulate griseofulvin compositions of the invention, when administered in the fed versus the fasted state, is less than about 100%, less than about 90%, less than about 80%, less than about 70%, less than about 60%, less than about 50%, less than about 40%, less than about 30%, less than about 20%, less than about 15%, less than about 10%, less than about 5%, less than about 3%, or essentially no difference.

Benefits of a dosage form that substantially eliminates the effect of food include an increase in subject convenience, thereby increasing subject compliance, as the subject does not need to ensure that they are taking a dose either with or without food.

D. Redispersibility Profiles of the Nanoparticulate Griseofulvin Compositions of the Invention

An additional feature of the nanoparticulate griseofulvin compositions of the invention is that the compositions redisperse such that the effective average particle size of the redispersed griseofulvin particles is less than about 2 microns. This is significant, as if upon administration the nanoparticulate griseofulvin particles present in the compositions of the invention did not redisperse to a substantially nanoparticulate particle size, then the dosage form may lose the benefits afforded by formulating griseofulvin into a nanoparticulate particle size.

This is because nanoparticulate griseofulvin compositions benefit from the small particle size of griseofulvin; if the nanoparticulate griseofulvin particles do not redisperse into the small particle sizes upon administration, then “clumps” or agglomerated griseofulvin particles are formed. With the formation of such agglomerated particles, the bioavailability of the dosage form may fall.

Moreover, the nanoparticulate griseofulvin compositions of the invention exhibit dramatic redispersion of the griseofulvin particles upon administration to a mammal, such as a human or animal, as demonstrated by reconstitution in a biorelevant aqueous media. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed griseofulvin particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

E. Bioadhesive Nanoparticulate Griseofulvin Compositions

Bioadhesive nanoparticulate griseofulvin compositions of the invention comprise at least one cationic surface stabilizer, which are described in more detail below. Bioadhesive formulations of griseofulvin exhibit exceptional bioadhesion to biological surfaces, such as mucous and skin.

In the case of bioadhesive nanoparticulate griseofulvin compositions, the term “bioadhesion” is used to describe the adhesion between the nanoparticulate griseofulvin compositions and a biological substrate (i.e., gastrointestinal mucin, lung tissue, nasal mucosa, etc.). See e.g., U.S. Pat. No. 6,428,814 for “Bioadhesive Nanoparticulate Compositions Having Cationic Surface Stabilizers,” which is specifically incorporated by reference.

The bioadhesive griseofulvin compositions of the invention are useful in any situation in which it is desirable to apply the compositions to a biological surface. The bioadhesive griseofulvin compositions preferably coat the targeted surface in a continuous and uniform film that is invisible to the naked human eye.

A bioadhesive nanoparticulate griseofulvin composition slows the transit of the composition, and some griseofulvin particles would also most likely adhere to tissue other than the mucous cells and therefore give a prolonged exposure to griseofulvin, thereby increasing absorption and the bioavailability of the administered dosage.

F. Low Viscosity

A liquid dosage form of a conventional microcrystalline or non-nanoparticulate griseofulvin composition would be expected to be a relatively large volume, highly viscous substance which would not be well accepted by patient populations. Moreover, viscous solutions can be problematic in parenteral administration because these solutions require a slow syringe push and can stick to tubing. In addition, conventional formulations of poorly water-soluble active agents, such as griseofulvin, tend to be unsafe for intravenous administration techniques, which are used primarily in conjunction with highly water-soluble substances.

Liquid dosage forms of the nanoparticulate griseofulvin compositions of the invention provide significant advantages over a liquid dosage form of a conventional microcrystalline or solubilized griseofulvin composition. The low viscosity and silky texture of liquid dosage forms of the nanoparticulate griseofulvin compositions of the invention result in advantages in both preparation and use. These advantages include, for example: (1) better subject compliance due to the perception of a lighter formulation which is easier to consume and digest; (2) ease of dispensing because one can use a cup or a syringe; (3) potential for formulating a higher concentration of griseofulvin resulting in a smaller dosage volume and thus less volume for the subject to consume; and (4) easier overall formulation concerns.

Liquid griseofulvin dosage forms that are easier to consume are especially important when considering juvenile patients, terminally ill patients, and elderly patients. Viscous or gritty formulations, and those that require a relatively large dosage volume, are not well tolerated by these patient populations. Liquid oral dosage forms can be particularly preferably for patient populations who have difficulty consuming tablets, such as infants and the elderly.

The viscosities of liquid dosage forms of nanoparticulate griseofulvin according to the invention are preferably less than about 1/200, less than about 1/175, less than about 1/150, less than about 1/125, less than about 1/100, less than about 1/75, less than about 1/50, or less than about 1/25 of a liquid oral dosage form of a non-nanoparticulate griseofulvin composition, at about the same concentration per ml of griseofulvin.

Typically the viscosity of liquid nanoparticulate griseofulvin dosage forms of the invention, at a shear rate of 0.1 (l/s) measured at 20° C., is from about 2000 mPa s to about 1 mPa s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 1 mPa·s, from about 500 mPa·s to about 1 mPa·s, from about 400 mPa·s to about 1 mPa·s, from about 300 mPa·s to about 1 mPa·s, from about 200 mPa·s to about 1 mPa·s, from about 175 mPa·s to about 1 mPa·s, from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s, from about 25 mPa·s to about 1 mPa·s, from about 15 mPa·s to about 1 mPa·s, from about 10 mPa·s to about 1 mPa·s, or from about 5 mPa·s to about 1 mPa·s. Such a viscosity is much more attractive for subject consumption and may lead to better overall subject compliance.

Viscosity is concentration and temperature dependent. Typically, a higher concentration results in a higher viscosity, while a higher temperature results in a lower viscosity. Viscosity as defined above refers to measurements taken at about 20° C. (The viscosity of water at 20° C. is 1 mPa s.) The invention encompasses equivalent viscosities measured at different temperatures.

Another important aspect of the invention is that the nanoparticulate griseofulvin compositions of the invention, formulated into a liquid dosage form, are not turbid. “Turbid,” as used herein refers to the property of particulate matter that can be seen with the naked eye or that which can be felt as “gritty.” The nanoparticulate griseofulvin compositions of the invention, formulated into a liquid dosage form, can be poured out of or extracted from a container as easily as water, whereas a liquid dosage form of a non-nanoparticulate or solubilized griseofulvin is expected to exhibit notably more “sluggish” characteristics.

The liquid formulations of this invention can be formulated for dosages in any volume but preferably equivalent or smaller volumes than a liquid dosage form of a non-nanoparticulate griseofulvin composition.

G. Sterile Filtered Nanoparticulate Griseofulvin Compositions

The nanoparticulate griseofulvin compositions of the invention can be sterile filtered. This obviates the need for heat sterilization, which can harm or degrade griseofulvin, as well as result in crystal growth and particle aggregation.

Sterile filtration can be difficult because of the required small particle size of the composition. Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize suspensions of micron-sized griseofulvin because the griseofulvin particles are too large to pass through the membrane pores.

A sterile nanoparticulate griseofulvin dosage form is particularly useful in treating immunocompromised patients, infants or juvenile patients, and the elderly, as these patient groups are the most susceptible to infection caused by a non-sterile liquid dosage form.

Because the nanoparticulate griseofulvin compositions of the invention, formulated into a liquid dosage form, can be sterile filtered, and because the compositions can have a very small griseofulvin effective average particle size, the compositions are suitable for parenteral administration.

H. Combination Pharmacokinetic Profile Compositions

In yet another embodiment of the invention, a first nanoparticulate griseofulvin composition providing a desired pharmacokinetic profile is co-administered, sequentially administered, or combined with at least one other griseofulvin composition that generates a desired different pharmacokinetic profile. More than two griseofulvin compositions can be co-administered, sequentially administered, or combined. While the first griseofulvin composition has a nanoparticulate particle size, the additional one or more griseofulvin compositions can be nanoparticulate, solubilized, or have a microparticulate particle size.

For example, a first griseofulvin composition can have a nanoparticulate particle size, conferring a short Tₘₐₓ and typically a higher Cₘₐₓ. This first griseofulvin composition can be combined, co-administered, or sequentially administered with a second composition comprising: (1) griseofulvin having a larger (but still nanoparticulate as defined herein) particle size, and therefore exhibiting slower absorption, a longer Tₘₐₓ, and typically a lower Cₘₐₓ; or (2) a microparticulate or solubilized griseofulvin composition, exhibiting a longer Tₘₐₓ, and typically a lower Cₘₐₓ.

The second, third, fourth, etc., griseofulvin compositions can differ from the first, and from each other, for example: (1) in the effective average particle sizes of griseofulvin; or (2) in the dosage of griseofulvin. Such a combination composition can reduce the dose frequency required.

If the second griseofulvin composition has a nanoparticulate particle size, then preferably the griseofulvin particles of the second composition have at least one surface stabilizer associated with the surface of the drug particles. The one or more surface stabilizers can be the same as or different from the surface stabilizer(s) present in the first griseofulvin composition.

Preferably where co-administration of a “fast-acting” formulation and a “longer-lasting” formulation is desired, the two formulations are combined within a single composition, for example a dual-release composition.

I. Combination Active Agent Compositions

The invention encompasses the nanoparticulate griseofulvin compositions of the invention formulated or co-administered with one or more non-griseofulvin active agents. Methods of using such combination compositions are also encompassed by the invention. The non-griseofulvin active agents can be present in a crystalline phase, an amorphous phase, a semi-crystalline phase, a semi-amorphous phase, or a mixture thereof.

The compound to be administered in combination with a nanoparticulate griseofulvin composition of the invention can be formulated separately from the nanoparticulate griseofulvin composition or co-formulated with the nanoparticulate griseofulvin composition. Where a nanoparticulate griseofulvin composition is co-formulated with a second active agent, the second active agent can be formulated in any suitable manner, such as immediate-release, rapid-onset, sustained-release, or dual-release form.

Such non-griseofulvin active agents can be, for example, a therapeutic agent. A therapeutic agent can be a pharmaceutical agent, including a biologic. The active agent can be selected from a variety of known classes of drugs, including, for example, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, such as NSAIDs and COX-2 inhibitors, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, alpha-adrenergic receptor blocking agents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Examples of representative active agents useful in this invention include, but are not limited to, acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

A description of these classes of active agents and a listing of species within each class can be found in Martindale's The Extra Pharmacopoeia, 31^{st} Edition (The Pharmaceutical Press, London, 1996), specifically incorporated by reference. The active agents are commercially available and/or can be prepared by techniques known in the art. Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., arginine, iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.” Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. Dietary supplements and nutraceuticals are also disclosed in Physicians' Desk Reference for Nutritional Supplements, 1st Ed. (2001) and The Physicians' Desk Reference for Herbal Medicines, 1st Ed. (2001), both of which are also incorporated by reference. A nutraceutical or dietary supplement, also known as a phytochemical or functional food, is generally any one of a class of dietary supplements, vitamins, minerals, herbs, or healing foods that have medical or pharmaceutical effects on the body.

In a preferred embodiment of the invention, the griseofulvin compositions of the invention are co-administered or combined with at least one other antifungal agent.

In another preferred embodiment of the invention, the griseofulvin compositions of the invention are co-administered or combined with vitamin E.

J. Miscellaneous Benefits of the Nanoparticulate Griseofulvin Compositions of the Invention

The nanoparticulate griseofulvin compositions preferably exhibit an increased rate of dissolution as compared to microcrystalline or non-nanoparticulate forms of griseofulvin. In addition, the nanoparticulate griseofulvin compositions preferably exhibit improved performance characteristics for oral, intravenous, subcutaneous, or intramuscular injection, such as higher dose loading and smaller tablet or liquid dose volumes. Moreover, the nanoparticulate griseofulvin compositions of the invention do not require organic solvents or pH extremes.

II. Griseofulvin Compositions

The invention provides compositions comprising nanoparticulate griseofulvin particles and at least one surface stabilizer. The surface stabilizers are preferably associated with the surface of the griseofulvin particles. Surface stabilizers useful herein do not chemically react with the griseofulvin particles or itself. Preferably, individual molecules of the surface stabilizer are essentially free of intermolecular cross-linkages. The compositions can comprise two or more surface stabilizers.

The present invention also includes nanoparticulate griseofulvin compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration (in solid, liquid, or aerosol (i.e., pulmonary) form), vaginal, nasal, rectal, ocular, local (powders, creams, ointments or drops), buccal, intracisternal, intraperitoneal, topical administration, and the like.

A. Griseofulvin Particles

Griseofulvin as used herein includes the compound having the chemical formula C₁₇H₁₇ClO₆ and the following chemical structure: 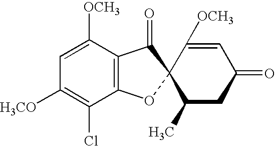
and salts thereof. See The Merck Index, 10^{th} Edition, pp. 4433-34 (1983).

Griseofulvin can be in a crystalline phase, an amorphous phase, a semi-crystalline phase, a semi-amorphous phase, or a mixture thereof.

B. Surface Stabilizers

The choice of a surface stabilizer for griseofulvin is non-trivial and required extensive experimentation to realize a desirable formulation. Accordingly, the present invention is directed to the surprising discovery that nanoparticulate griseofulvin compositions can be made.

Combinations of more than one surface stabilizer can be used in the invention. Useful surface stabilizers that can be employed in the invention include, but are not limited to, known organic and inorganic pharmaceutical excipients. Such excipients include various polymers, low molecular weight oligomers, natural products, and surfactants. Surface stabilizers include nonionic, anionic, cationic, zwitterionic, and ionic surfactants.

Representative examples of other useful surface stabilizers include hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

Depending upon the desired method of administration, bioadhesive formulations of nanoparticulate griseofulvin can be prepared by selecting one or more cationic surface stabilizers that impart bioadhesive properties to the resultant composition. Useful cationic surface stabilizers are described below.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt) (also known as DPPE-PEG(2000)-Amine Na) (Avanti Polar Lipids, Alabaster, Ala.), Poly(2-methacryloxyethyl trimethylammonium bromide) (Polysciences, Inc., Warrington, Pa.) (also known as S1001), poloxamines such as Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX (Dow, Midland, Mich.).

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)₄ ammonium chloride or bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric cationic surface stabilizers are any nonpolymeric compound, such as benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁—R₄ are CH₃;
(ii) one of R₁—R₄ is CH₃;
(iii) three of R₁—R₄, are CH₃;
(iv) all of R₁—R₄ are CH₃;
(v) two of R₁—R₄ are CH₃, one of R₁—R₄ is C₆H₅CH₂, and one of R₁—R₄ is an alkyl chain of seven carbon atoms or less;
(vi) two of R₁—R₄ are CH₃, one of R₁—R₄ is C₆H₅CH₂, and one of R₁—R₄ is an alkyl chain of nineteen carbon atoms or more;
(vii) two of R₁—R₄ are CH₃ and one of R₁—R₄ is the group C₆H₅(CH₂)ₙ, where n>1;
(viii) two of R₁—R₄ are CH₃, one of R₁—R₄ is C₆H₅CH₂, and one of R₁—R₄ comprises at least one heteroatom;
(ix) two of R₁—R₄ are CH₃, one of R₁—R₄ is C₆H₅CH₂, and one of R₁—R₄ comprises at least one halogen;
(x) two of R₁—R₄ are CH₃, one of R₁—R₄ is C₆H₅CH₂, and one of R₁—R₄ comprises at least one cyclic fragment;
(xi) two of R₁—R₄ are CH₃ and one of R₁—R₄ is a phenyl ring; or
(xii) two of R₁—R₄ are CH₃ and two of R₁—R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

In a preferred embodiment, the compositions of the invention do not contain as a surface stabilizer a polyalkylene block copolymer which gels at physiological temperature, and which contains one or more polyoxyethylene blocks and one or more polyoxy (higher alkylene) blocks, wherein at least some of the blocks are linked together by an oxymethylene group. Such compounds are described in U.S. Pat. No. 5,705,194.

Preferred surface stabilizers include, but are not limited to, Tween® 80, Tetronic® T908, dioctylsulfosuccinate, polyvinyl acetate, Pluronic® F127, or a mixture thereof.

Tween® 80 is polyoxyethylene sorbitan monooleate(polyoxyethylenesorbitan monooleat). Tetronic® T908 is a tetrafunctional block copolymer derived from sequential addition of ethylene oxide and propylene oxide to ethylene-diamine available from BASF. Pluronic® F127, which is a poloxamer, is a difunctional block copolymer surfactant of ethylene oxide and propylene oxide terminating in primary hydroxyl groups and having a molecular weight of about 12,600 daltons (see

http://www.basf.com/businesses/chemicals/performance/pdfs/Pluronic F1 27.pdf. Pluronic® F127 has the chemical formula of PEO₁₀₀PPO₆₅PEO₁₀₀.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

C. Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

D. Nanoparticulate Griseofulvin Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The compositions of the invention comprise griseofulvin nanoparticles which have an effective average particle size of less than about 2000 nm (i.e., 2 microns), less than about 1900 nm, less than less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, or less than about 50 nm, when measured by the above-noted techniques.

By “an effective average particle size of less than about 2000 nm” it is meant that at least 50% of the nanoparticulate griseofulvin particles have a weight average particle size less than about 2000 nm, when measured by the above-noted techniques. In other embodiments of the invention, at least about 70%, at least about 90%, at least about 95%, or at least about 99% of the griseofulvin particles have a particle size less than the effective average, by weight, i.e., less than about 2000 nm, less than about 1900 nm, less than less than about 1800 nm, less than about 1700 nm, etc.

If the nanoparticulate griseofulvin composition is combined with a microparticulate griseofulvin or non-griseofulvin active agent composition, then such a composition is either solubilized or has an effective average particle size greater than about 2 microns. By “an effective average particle size of greater than about 2 microns” it is meant that at least 50% of the microparticulate griseofulvin or non-griseofulvin active agent particles have a particle size greater than about 2 microns, by weight, when measured by the above-noted techniques. In other embodiments of the invention, at least about 70%, at least about 90%, at least about 95%, or at least about 99%, by weight, of the microparticulate griseofulvin or non-griseofulvin active agent particles have a particle size greater than about 2 microns.

In the present invention, the value for D50 of a nanoparticulate griseofulvin composition is the particle size below which 50% of the griseofilvin particles fall, by weight. Similarly, D90 and D99 are the particle sizes below which 90% and 99%, respectively, of the griseofulvin particles fall, by weight.

E. Concentration of Nanoparticulate Griseofulvin and Surface Stabilizers

The relative amounts of griseofulvin and one or more surface stabilizers can vary widely. The optimal amount of the individual components can depend, for example, upon the hydrophilic lipophilic balance (HLB), melting point, and the surface tension of water solutions of the stabilizer, etc.

The concentration of griseofulvin can vary from about 99.5% to about 0.001%, from about 95% to about 0.1%, or from about 90% to about 0.5%, by weight, based on the total combined dry weight of the griseofulvin and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the griseofulvin and at least one surface stabilizer, not including other excipients.

III. Methods of Making Nanoparticulate Griseofulvin Formulations

The nanoparticulate griseofulvin compositions can be made using, for example, milling, homogenization, or precipitation techniques. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

Following milling, homogenization, precipitation, etc., the resultant nanoparticulate griseofulvin composition can be utilized in solid or liquid dosage formulations, such as controlled release formulations, solid dose fast melt formulations, aerosol formulations, nasal formulations, lyophilized formulations, tablets, capsules, solid lozenge, powders, creams, ointments, etc.

A. Milling to Obtain Nanoparticulate Griseofulvin Dispersions

Milling griseofulvin to obtain a nanoparticulate dispersion comprises dispersing griseofulvin particles in a liquid dispersion media in which griseofulvin is poorly soluble, followed by applying mechanical means in the presence of grinding media to reduce the particle size of griseofulvin to the desired effective average particle size. The dispersion media can be, for example, water, safflower oil, ethanol, t-butanol, glycerin, polyethylene glycol (PEG), hexane, or glycol.

The griseofulvin particles can be reduced in size in the presence of at least one surface stabilizer. Alternatively, the griseofulvin particles can be contacted with one or more surface stabilizers after attrition. Other compounds, such as a diluent, can be added to the griseofulvin/surface stabilizer composition during the size reduction process. Dispersions can be manufactured continuously or in a batch mode.

B. Precipitation to Obtain Nanoparticulate Griseofulvin Compositions

Another method of forming the desired nanoparticulate griseofulvin composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving griseofulvin in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

C. Homogenization to Obtain Nanoparticulate Griseofulvin Compositions

Exemplary homogenization methods of preparing active agent nanoparticulate compositions are described in U.S. Pat. No. 5,510,118, for “Process of Preparing Therapeutic Compositions Containing Nanoparticles.”

Such a method comprises dispersing griseofulvin particles in a liquid dispersion media in which griseofulvin is poorly soluble, followed by subjecting the dispersion to homogenization to reduce the particle size of the griseofulvin to the desired effective average particle size. The dispersion media can be, for example, water, safflower oil, ethanol, t-butanol, glycerin, polyethylene glycol (PEG), hexane, or glycol.

The griseofulvin particles can be reduced in size in the presence of at least one surface stabilizer. Alternatively, the griseofulvin particles can be contacted with one or more surface stabilizers either before or after attrition. Other compounds, such as a diluent, can be added to the griseofulvin/surface stabilizer composition either before, during, or after the size reduction process. Dispersions can be manufactured continuously or in a batch mode.

IV. Methods of Using Nanoparticulate Griseofulvin Formulations

The method of the invention comprises administering to a subject an effective amount of a composition comprising nanoparticulate griseofulvin. The griseofulvin compositions of the present invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

The griseofulvin compositions of the invention can be used to treat dermatophyte and ringworm infections of the scalp, hair, nails (fingernails and toenails), and skin. The griseofulvin compositions can be used to treat skin infections such as jock itch, athlete's foot, and ringworm. In particular, the griseofulvin compositions of the invention can be used to treat Tinea capitis (ringworm of the scalp), Tinea corporis (ringworm of the body), Tinea pedis (athlete's foot), Tinea unguium (ringworm of the nails), Tinea cruris (ringworm of the thigh), and Tinea barbae (barber's itch). The griseofulvin compositions of the invention can also used to the following fungal infections of the hair, skin, and nails: Trichophyton rubrum, Trichophyton tonsurans, Trichophyton mentagrophytes, Trichophyton interdigitalis, Trichophyton verrucosum, Trichophyton sulphureum, Trichophyton schoenleini, Trichophyton audouini, Trichophyton canis, Trichophyton gypseum, Trichophyton floccosum, Trichophyton megnini, Trichophyton gallinae, and Trichophyton crateriform.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, powder aerosols, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable aerosols, emulsions, solutions, suspensions, syrups, and elixirs. In addition to the active agent, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

One of ordinary skill will appreciate that effective amounts of griseofulvin can be determined empirically and can be employed in pure form or, where such forms exist, in pharmaceutically acceptable salt, ester, or prodrug form. Actual dosage levels of griseofulvin in the nanoparticulate compositions of the invention may be varied to obtain an amount of griseofulvin that is effective to obtain a desired therapeutic response for a particular composition and method of administration. The selected dosage level therefore depends upon the desired therapeutic effect, the route of administration, the potency of the administered griseofulvin, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLE 1

The purpose of this example was to prepare a nanoparticulate griseofulvin composition.

5.0% (w/w) griseofulvin and 2.5% (w/w) Pluronic® F68 were combined in an aqueous media (water). 3.75 mLs of this mixture was then charged into a ½ oz bottle (15 mL) for roller milling on a Bench top roller mill (US Stoneware, East Palestine, Ohio) along with 1.0 mm zirconium oxide milling media. The griseofulvin slurry was then milled for 2 days.

Following milling, the D50 particle size of the griseofulvin particles was 617 nm, and the D90 was 1000 nm. Particle size was determined on the Coulter Model N4MD Submicron Particle Analyzer (Coulter Corp., Miami Lakes, Fla.), and using the Microtrac Ultrafine Particle Analyzer (Leeds and Northrup Co., St. Petersburg, Fla.).

This example demonstrates that nanoparticulate compositions of griseofulvin can be made.

### EXAMPLE 2

The purpose of this example was to prepare a nanoparticulate griseofulvin composition.

5.0% (w/w) griseofulvin and 2.5% (w/w) Pluronic® F127 were combined in an aqueous media (water). 3.75 mLs of this mixture was then charged into a ½ oz bottle (15 mL) for roller milling on a Bench top roller mill (US Stoneware, East Palestine, Ohio) along with 1.0 mm zirconium oxide milling media. The griseofulvin slurry was then milled for 5 days.

Following milling, the D90 particle size of the griseofulvin particles was 464 nm. Particle size was determined on the Coulter Model N4MD Submicron Particle Analyzer (Coulter Corp., Miami Lakes, Fla.), and using the Microtrac Ultrafine Particle Analyzer (Leeds and Northrup Co., St. Petersburg, Fla.).

This example demonstrates that nanoparticulate compositions of griseofulvin can be made.

### EXAMPLE 3

The purpose of this example was to prepare a pharmaceutical composition utilizing the nanoparticulate griseofulvin composition of Example 2.

The nanoparticulate griseofulvin composition of Example 2 was combined with pharmaceutical excipients and carriers as shown below in Table 1.

TABLE 1

| Ingredient | Quantity | |
|---|---|---|
| Griseofulvin | 5.0 | g |
| Pluronic F127 | 2.5 | g |
| Benzoate Sodium | 0.2 | g |
| Saccharin Sodium | 0.1 | g |
| FD&C Red. No. 3 | 0.03 | g |
| Water, qs | 100 | mL |

This example demonstrates the successful preparation of a pharmaceutical composition comprising a nanoparticulate griseofulvin composition.

### EXAMPLE 4

The purpose of this example was to prepare nanoparticulate griseofulvin compositions using various surface stabilizers.

An aqueous slurry of 5% (w/w) griseofulvin and 2.5% surface stabilizer(s) in water was prepared. The surface stabilizer(s) are identified in Table 2, below. 3.75 mLs of each slurry was then charged into a ½ oz bottle (15 mL) for roller milling on a Bench top roller mill (US Stoneware, East Palestine, Ohio) along with 1.0 mm zirconium oxide milling media. Each slurry was then milled for the time period shown in Table 2.

Following completion of milling, the average particle size, by weight, of the griseofulvin was determined. In addition, the D80 and/or D90 griseofulvin particle sizes were determined. Particle size was determined on the Coulter Model N4MD Submicron Particle Analyzer (Coulter Corp., Miami Lakes, Fla.), and using the Microtrac Ultrafine Particle Analyzer (Leeds and Northrup Co., St. Petersburg, Fla.).

TABLE 2

| | | Weight Average | | |
|---|---|---|---|---|
| | | Griseofulvin | | |
| | Milling | Particle | | |
| Surface Stabilizer and | Time | Size (D50) | D80 | D90 |
| Quantity Thereof | (days) | (nm) | (nm) | (nm) |
| 2.5% polyvinyl acetate | 2 | 459 | | 464 |
| 70-100K (PVA) | | | | |
| 2.5% PVA 70-100K + | 10 | 276 | 215 | 464 |
| 2.5% dioctylsulfo- | | | | |
| succinate (DOSS) | | | | |
| 2.5% DOSS | 2 | 225 | | 215 |
| 2.5% DOSS 2.5% DOSS | 10 | 224 | 215 | 464 |
| 2.5% Tetronic ® 908 | 2 | 101 | | 100 |
| 2.5% Tetronic ® 908 + | 10 | 310 | | 464 |
| 2.5% DOSS | | | | |
| 2.5% Pluronic ® F127 | 5 | 519 | | 464 |
| 2.5% Pluronic ® F127 + | 10 | 309 | | 2150 |
| 2.5% DOSS | | | | |
| 2.5% tyloxapol | 5 | 492 | 215 | 464 |
| 2.5% tyloxapol + | 6 | 279 | 215 | 464 |
| 2.5% DOSS | | | | |
| 2.5% Tween ® 80 | 6 | 290 | 215 | 464 |
| 2.5% Tween ® 80 + | 6 | 282 | 215 | 464 |
| 2.5% DOSS | | | | |
| 2.5% Pharmacoat ® 603 | 6 | 77.5 | | 10,000 |
| 2.5% Pharmacoat ® 603 + | 2 | 438 | 464 | 1000 |
| 2.5% DOSS | | | | |
| 2.5% Pluronic ® F68 | 6 | 617 | | 1000 |
| 2.5% Pluronic ® F68 + | 2 | 864 | | 10,000 |
| 2.5% DOSS | | | | |
| 2.5% Polyvinylpyrrolidone | 2 | 455 | | 10,000 |
| K15 (PVP) | | | | |
| 2.5% PVP K15 + | 10 | 270 | 215 | 464 |
| 2.5% DOSS | | | | |

All of the milled compositions were tested for stability in water, simulated gastric fluid (SGF), and simulated intestinal fluid (SIF) (SGF and SIF were prepared according to the USP). In addition, the stability of the milled compositions following storage for one week at room temperature was determined.

The particle morphology of griseofulvin appears to be spherical in nature when observed by light microscopy.

Based on the above data, and the lack of aggregation in fluid stability tests and stable one week shelf stabilities, the following surface stabilizers for Nanoparticulate griseofulvin are preferred:

(1) Tween® 80: This surface stabilizer is Generally Recognized as Safe (GRAS) (see “Inactive Ingredient Guide, Division of Drug Information Resources Food and Drug Administration, Center for Drug Evaluation and Research, Office of Management, January 1996). In addition, nanoparticulate griseofulvin compositions utilizing this surface stabilizer exhibited small size particles and were stable in water, simulated gastric fluid, and simulated intestinal fluid;

(2) Tetronic® T908: This surface stabilizer is GRAS, and nanoparticulate griseofulvin compositions utilizing this surface stabilizer exhibited small size particles and were stable in water, simulated gastric fluid, and simulated intestinal fluid; and

(3) Dioctylsulfosuccinate: This surface stabilizer is GRAS, and nanoparticulate griseofulvin compositions utilizing this surface stabilizer exhibited small size particles and were stable in water, simulated gastric fluid, and simulated intestinal fluid.

Less preferred but still useful are nanoparticulate griseofulvin compositions comprising as a surface stabilizer polyvinyl acetate or Pluronic® F127.

The following surface stabilizers performed poorly during the milling assay, and exhibited poor stability in water, simulated gastric fluid, and/or simulated intestinal fluid:

(1) Pharmacoat® 603: A bimodal griseofulvin particle size distribution and large griseofulvin particles were observed;

(2) Pluronic® F68: A bimodal griseofulvin particle size distribution was observed;

(3) Ponlyvinylpyrrolidone: The griseofulvin composition comprising PVP as a surface stabilizer exhibited poor stability in water, simulated gastric fluid, and simulated intestinal fluid; and

(4) Tyloxapol: The griseofulvin composition comprising tyloxapol as a surface stabilizer exhibited poor stability in water, simulated gastric fluid, and simulated intestinal fluid.

While the latter four surface stabilizers were not successful in producing a stable nanoparticulate griseofulvin composition in this experiment, the surface stabilizers may be useful at different concentrations of griseofulvin or surface stabilizer, or when used in combination with one or more other surface stabilizers.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1.-41. (canceled)

42. A method of making a griseofulvin composition comprising contacting particles of griseofulvin or a salt thereof with at least one surface stabilizer for a time and under conditions sufficient to provide a griseofulvin composition having an effective average particle size of less than about 2000 nm, wherein the surface stabilizer is not a polyalkylene block copolymer which gels at physiological temperature, and which contains one or more polyoxyethylene blocks and one or more polyoxy (higher alkylene) blocks, wherein at least some of the blocks are linked together by an oxymethylene group.

43. The method of claim 42, wherein said contacting comprises grinding, wet grinding, homogenizing, precipitation, or a combination thereof.

44.-46. (canceled)

47. The method of claim 42, wherein the griseofulvin or a salt thereof is selected from the group consisting of a crystalline phase, an amorphous phase, a semi-crystalline phase, a semi-amorphous phase, and mixtures thereof.

48. The method of claim 42, wherein the effective average particle size of the griseofulvin particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

49. The method of claim 42, wherein the composition is formulated:
(a) for administration selected from the group consisting of oral, pulmonary, rectal, opthalmic, colonic, parenteral, intracisternal, intravaginal, intraperitoneal, local, buccal, nasal, and topical administrations
(b) into dosage form selected from the group consisting of liquid dispersions, oral suspensions, gels, aerosols, ointments, creams, tablets, and capsules;
(c) into a dosage form selected from the group consisting of controlled release formulations, fast melt formulations, lyophilized formulations, delayed release formulations, extended release formulations, pulsatile release formulations, and mixed immediate release and controlled release formulations; or
(d) a combination thereof.

50. The method of claim 42, wherein the composition further comprises one or more pharmaceutically acceptable excipients, carriers, or a combination thereof.

51. The method of claim 42, wherein:
(a) griseofulvin or a salt thereof is present in an amount selected from the group consisting of from about 99.5% to about 0.001%, from about 95% to about 0.1%, and from about 90% to about 0.5%, by weight, based on the total combined dry weight of the griseofulvin or a salt thereof and at least one surface stabilizer, not including other excipients;
(b) at least one surface stabilizer is present in an amount selected from the group consisting of from about 0.5% to about 99.999%, from about 5.0% to about 99.9%, and from about 10% to about 99.5% by weight, based on the total combined dry weight of the griseofulvin or a salt thereof and at least one surface stabilizer, not including other excipients; or
(c) a combination thereof.

52. (canceled)

53. The method of claim 42, utilizing at least two surface stabilizers.

54. The method of claim 42, wherein:
a the surface stabilizer is selected from the group consisting of an anionic surface stabilizer, a cationic surface stabilizer, a zwitterionic surface stabilizer, and an ionic surface stabilizers;
(b) at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, Polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone;
(c) at least one cationic surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar;
(d) the composition comprises as a surface stabilizer Tween® 80, Tetronic® T908, dioctylsulfosuccinate, polyvinyl acetate, Pluronic® F127, or a mixture thereof; or
(d) any combination thereof.

55.-57. (canceled)

58. The method of claim 54, wherein the composition is bioadhesive.

59. (canceled)

60. A method of treating a subject in need comprising administering to the subject an effective amount of a composition comprising:
(a) particles of a griseofulvin or a salt thereof, wherein the griseofulvin particles have an effective average particle size of less than about 2000 nm; and
(b) at least one surface stabilizer, wherein the surface stabilizer is not a polyalkylene block copolymer which gels at physiological temperature, and which contains one or more polyoxyethylene blocks and one or more polyoxy (higher alkylene) blocks, wherein at least some of the blocks are linked together by an oxymethylene group.

61. The method of claim 60, wherein the griseofulvin or a salt thereof is selected from the group consisting of a crystalline phase, an amorphous phase, a semi-crystalline phase, a semi-amorphous phase, and mixtures thereof.

62. The method of claim 60, wherein the effective average particle size of the griseofulvin particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

63. The method of claim 60, wherein the composition is formulated:
(a) for administration selected from the group consisting of oral, pulmonary, rectal, opthalmic, colonic, parenteral, intracisternal, intravaginal, intraperitoneal, local, buccal, nasal, and topical administrations;
(b) into a dosage form selected from the group consisting of liquid dispersions, oral suspensions, gels, aerosols, ointments, creams, tablets, and capsules;
(c) into a dosage form selected from the group consisting of controlled release formulations, fast melt formulations, lyophilized formulations, delayed release formulations, extended release formulations, pulsatile release formulations, and mixed immediate release and controlled release formulations; or
(d) a combination thereof.

64. (canceled)

65. The method of claim 60, wherein the composition further comprises one or more pharmaceutically acceptable excipients, carriers, or a combination thereof.

66. The method of claim 60, wherein:
(a) griseofulvin or a salt thereof is present in an amount selected from the group consisting of from about 99.5% to about 0.001%, from about 95% to about 0.1%, and from about 90% to about 0.5%, by weight, based on the total combined dry weight of the griseofulvin or a salt thereof and at least one surface stabilizer, not including other excipients;
(b) at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the griseofulvin or a salt thereof and at least one surface stabilizer, not including other excipients; or
(c) a combination thereof.

67. (canceled)

68. The method of claim 60, utilizing at least two surface stabilizers.

69. The method of claim 60, wherein:
(a) the surface stabilizer is selected from the group consisting of an anionic surface stabilizer, a cationic surface stabilizer, a zwitterionic surface stabilizer, and an ionic surface stabilizer;
(b) at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl P-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone;
(c) at least one cationic surface stabilizer is selected from the group consisting of a benzalkonium chloride, polymethylmethacrylate trimethylammonium bromide, polyvinylpyrrolidone-2-dimethylaminoethyl methacrylate dimethyl sulfate, hexadecyltrimethyl ammonium bromide, cationic lipids, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar;
(d) the composition comprises as a surface stabilizer Tween® 80, Tetronic® T908, dioctylsulfosuccinate, polyvinyl acetate, Pluronic® F127, or a mixture thereof; or
(e) any combination thereof.

70.-72. (canceled)

73. The method of claim 69, wherein the composition is bioadhesive.

74. (canceled)

75. The method of claim 60, additionally comprising administering one or more non-griseofulvin active agents.

76. The method of claim 75, wherein:
(a) said additional one or more non-griseofulvin active agents are selected from the group consisting of nutraceuticals, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, alpha-adrenergic receptor blocking agents, beta-adrenoceptor blocking agents, blood products, blood substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, parathyroid biphosphonates, prostaglandins, radio-pharmaceuticals, sex hormones, anti-allergic agents, stimulants, anoretics, sympathomimetics, thyroid agents, vasodilators, and xanthines;
(b) at least one additional non-griseofulvin active agent is an anti-fungal agent;
(c) at least one additional non-griseofulvin active agent is vitamin E; or
(d) any combination thereof.

77.-78. (canceled)

79. The method of claim 60, wherein:
(a) the Tₘₐₓ of the griseofulvin, when assayed in the plasma of a mammalian subject following administration, is less than the Tₘₐₓ for a non-nanoparticulate griseofulvin formulation, administered at the same dosage;
(b) the Cₘₐₓ of the griseofulvin, when assayed in the plasma of a mammalian subject following administration, is greater than the Cₘₐₓ for a non-nanoparticulate griseofulvin formulation, administered at the same dosage;
(c) the AUC of the griseofulvin, when assayed in the plasma of a mammalian subject following administration, is greater than the AUC for a non-nanoparticulate griseofulvin formulation, administered at the same dosage; or
(d) a combination thereof.

80. The method of claim 79, wherein:
(a) the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate griseofulvin formulation, administered at the same dosage;
(b) the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate formulation of griseofulvin, administered at the same dosage;
(c) the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of griseofulvin, administered at the same dosage; or
(d) a combination thereof.

81.-84. (canceled)

85. The method of claim 60, wherein the griseofulvin composition does not produce significantly different absorption levels when administered under fed as compared to fasting conditions.

86. The method of claim 85, wherein the difference in absorption of the griseofulvin composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

87. The method of claim 60, wherein administration of the composition to a human in a fasted state is bioequivalent to administration of the composition to a human in a fed state.

88. The method of claim 87, wherein “bioequivalency” is established by:
(a) a 90% Confidence Interval of between 0.80 and 1.25 for both Cₘₐₓ and AUC;
(b) a 90% Confidence Interval of between 0.80 and 1.25 for AUC and a 90% Confidence Interval of between 0.70 to 1.43 for Cₘₐₓ; or
(c) a combination thereof.

89. (canceled)

90. The method of claim 60, wherein the subject is a human.

91. The method of claim 60, used to treat a condition selected from the group consisting of:
(a) dermatophyte infections and ringworm infections;
(b) ringworm infections of the scalp, hair, nails, and skin;
(c) Tinea capitis (ringworm of the scalp), Tinea corporis (ringworm of the body), Tinea pedis (athlete's foot), Tinea unguium (ringworm of the nails), Tinea cruris (ringworm of the thigh), and Tinea barbae (barber's itch);
(d) a fungal infection of an organism selected from the group consisting of Trichophyton rubrum, Trichophyton tonsurans, Trichophyton mentagrophytes, Trichophyton interdigitalis, Trichophyton verrucosum, Trichophyton sulphureum, Trichophyton schoenleini, Trichophyton audouini, Trichophyton canis, Trichophyton gypseum, Trichophyton floccosum, Trichophyton megnini, Trichophyton gallinae, and Trichophyton crateriform; or
(e) a combination thereof.

92.-94. (canceled)
